(19) United States
(12) Patent Application Publication
(10) Pub. No.: US 2011/0208048 A1
(43) Pub. Date: Aug. 25, 2011

(54) RADIATION IMAGING SYSTEM AND PROCESSING METHOD THEREFOR

(75) Inventor: Keisuke Arima, Yokohama-shi (JP)

(73) Assignee: CANON KABUSHIKI KAISHA, Tokyo (JP)

(21) Appl. No.: 13/012,365

(22) Filed: Jan. 24, 2011

(30) Foreign Application Priority Data
Feb. 22, 2010 (JP) 2010-036666

(51) Int. Cl. A61B 6/00; G06K 9/00

(52) U.S. Cl. 600/436; 382/132

(57) ABSTRACT

A radiation imaging system which captures a radiographic image obtains examination information including the patient information of a patient as a target for the imaging and imaging information defining the contents of imaging executed for the patient, controls the radiation imaging of a patient in accordance with the examination information, stores the captured radiographic image in association with the patient information of the image, and switches and displays, within one window, an imaging screen used for imaging based on the examination information and a playback screen used for playback of the stored radiographic image. The apparatus then detects a switching instruction for display of a displayed screen based on input operation by an operator, compares patient information corresponding to the imaging screen with patient information corresponding to the playback screen when display switching is performed based on the switching instruction, and notifies the operator of the comparison result.

### BACKGROUND OF THE INVENTION

1. Field of the Invention

The present invention relates to a radiation imaging system and a processing method for the system.

2. Description of the Related Art

Conventionally, there are known radiation imaging apparatuses which capture a radiographic image of an object by irradiating the object with radiation (for example, X-rays) and detecting the intensity distribution of the radiation transmitted through the object. Such apparatuses are mainly used in the medical field, industrial field, and the like. Recently, there is widely used a technique of obtaining a radiographic image as digital data by converting the radiation transmitted through an object into an electrical signal.

For example, in the medical field, examinations using radiation are routinely practiced. In general, for an examination using radiation (radiation examination), a doctor in each diagnosis and treatment department creates an examination request form which describes an imaging region and an imaging method. An imaging technician executes a radiation examination in accordance with the examination request form. Some hospitals have an intra-hospital network to which various kinds of medical devices are connected. In such a network environment, a radiation imaging system performs imaging in cooperation with a hospital information system (HIS), radiology information system (RIS), image server (PACS), and the like.

In a hospital in which an intra-hospital network is constructed, a doctor issues an examination request from the HIS instead of issuing an examination request form. In this case, the technician in the radiology department receives the examination request via the RIS, and executes a radiation examination using a radiation imaging system in accordance with the examination request.

Upon completion of the radiation examination, the radiation imaging system notifies the HIS and RIS of the completion of the examination, and outputs captured images and examination information to the PACS, printer, and the like. This system sometimes displays such captured images and examination information on a display device.

In association with this technique, Japanese Patent Laid-Open No. 2006-81761 has proposed a screen control technique of switching between an imaging screen at the time of the execution of an examination and a playback screen to display captured images upon completion of the examination by using tabs. This technique allows the user to switch between the imaging screen and the playback screen with one operation, and hence allows the user to easily compare and refer to examination images during and after the execution of the examination.

In general, images after the execution of an examination are compared with examination images during the examination to observe the progression of treatment of the same patient. When executing image processing for the purpose of obtaining an image suitable for the diagnosis made by the doctor, image parameters for the same region as that imaged in the past are sometimes used for reference. For this reason, the technique disclosed in Japanese Patent Laid-Open No. 2006-81761 is a highly convenient and useful technique. Although this technique allows easy comparison and reference, it increases the risk of mixing up patients.

### SUMMARY OF THE INVENTION

The present invention provides a technique of effectively prompting an operator to check a patient at the time of display switching.

According to a first aspect of the present invention there is provided a radiation imaging system which captures a radiographic image, the system comprising: an obtaining unit configured to obtain examination information including patient information of a patient as a target for the imaging and imaging information defining a content of imaging executed for the patient; an imaging control unit configured to control imaging of a patient using radiation in accordance with examination information obtained by the obtaining unit; a storage unit configured to store the captured radiographic image in association with the patient information of the image; a display unit configured to switch and display, within one window, an imaging screen used for imaging based on the examination information and a playback screen used for playback of a radiographic image stored in the storage unit; an instruction detection unit configured to detect a switching instruction for display of a screen displayed on the display unit, based on input operation by an operator; a comparison unit configured to compare patient information corresponding to the imaging screen with patient information corresponding to the playback screen when display switching is performed based on the switching instruction; and a notification unit configured to notify the operator of a comparison result obtained by the comparison unit.

According to a second aspect of the present invention there is provided a processing method in a radiation imaging system which captures a radiographic image, the method comprising: obtaining examination information including patient information of a patient as a target for the imaging and imaging information defining a content of imaging executed for the patient; controlling imaging of a patient using radiation in accordance with the obtained examination information; storing the captured radiographic image in association with the patient information of the image; switching and displaying, within one window, an imaging screen used for imaging based on the examination information and a playback screen used for playback of the stored radiographic image; detecting a switching instruction for display of the displayed screen based on input operation by an operator; comparing patient information corresponding to the imaging screen with patient information corresponding to the playback screen when display switching is performed based on the switching instruction; and notifying the operator of the comparison result.

Further features of the present invention will be apparent from the following description of exemplary embodiments (with reference to the attached drawings).

### BRIEF DESCRIPTION OF THE DRAWINGS

The accompanying drawings, which are incorporated in and constitute a part of the specification, illustrate embodiments of the invention, and together with the description, serve to explain the principles of the invention.

FIG. 1 is a view showing an example of the arrangement of a radiation imaging system (X-ray imaging system) according to an embodiment of the present invention;

FIG. 2 is a view showing an example of a screen layout;

FIGS. 3A and 3B are views each showing an example of a screen layout;

FIG. 4 is a view showing an example of a screen layout;

FIG. 5 is a view showing an example of a screen layout;

FIG. 6 is a block diagram showing an example of the functional arrangement of a control unit 13 shown in FIG. 1;

FIG. 7 is a flowchart showing an example of the operation of the radiation imaging system shown in FIG. 1;

FIG. 8 is a view showing an example of a screen layout;

FIG. 9 is a view showing an example of a screen layout; and

FIGS. 10A and 10B are views each showing an example of a screen layout.

### DESCRIPTION OF THE EMBODIMENTS

An exemplary embodiment(s) of the present invention will now be described in detail with reference to the drawings. It should be noted that the relative arrangement of the components, the numerical expressions and numerical values set forth in these embodiments do not limit the scope of the present invention unless it is specifically stated otherwise.

The following embodiments will exemplify a case in which X-rays are used as radiation. However, radiation is not limited to X-rays but may be electromagnetic waves, α-rays, β-rays, or γ-rays.

### First Embodiment

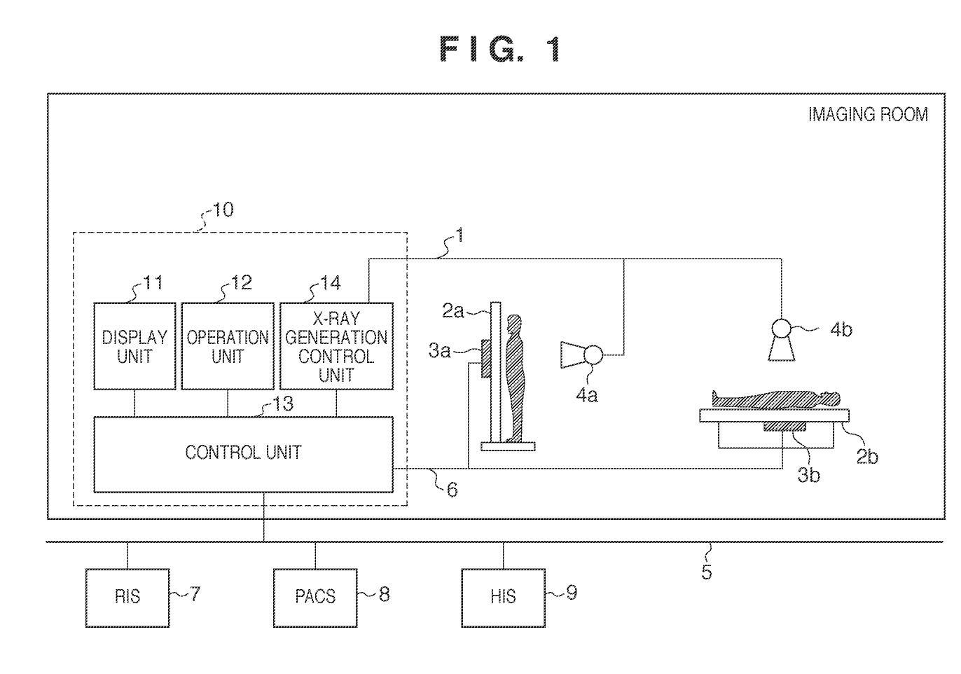

FIG. 1 is a view showing an example of the arrangement of a radiation imaging system (to be referred to as an X-ray imaging system) according to an embodiment of the present invention.

In the X-ray imaging system, an X-ray imaging apparatus 10, an RIS (Radiology Information System) 7, a PACS (image server) 8, and an HIS (Hospital Information System) 9 are connected to each other. These apparatuses are connected to each other via a communication unit 5 including a LAN (Local Area Network) or WAN (Wide Area Network). Note that each of these apparatuses includes one or a plurality of computers. Each computer includes, for example, a main control unit such as a CPU, a ROM (Read Only Memory), and a RAM (Random Access Memory). Each computer may also include a communication unit such as a network card and input and output units such as a keyboard, display, and touch panel. These components are connected via a bus. The main control unit controls them by executing programs stored in a storage unit.

In this case, the X-ray imaging apparatus 10 includes a display unit 11, an operation unit 12, a control unit 13, and an X-ray generation control unit 14.

The X-ray generation control unit 14 is connected to an X-ray generating unit 4, that is, 4a and 4b, via a cable 1, and controls the irradiation of X-rays from the X-ray generating unit 4. In this case, the X-ray generating units 4a and 4b each function as a radiation generating unit. The X-ray generating units 4a and 4b each are implemented by an X-ray tube, which irradiates an object (for example, a specific region of a patient) with X-rays.

The control unit 13 comprehensively controls processing in the X-ray imaging apparatus 10. The display unit 11 is implemented by, for example, a liquid crystal display, and displays various kinds of information to an operator (an imaging technician or doctor). The operation unit 12 is implemented by, for example, a mouse and operation buttons, and inputs various kinds of instructions from the operator into the apparatus. Note that the display unit 11 and the operation unit 12 may be implemented by a touch panel obtained by incorporating them.

The X-ray imaging apparatus 10 is connected to each of X-ray detection units 3, that is, 3a and 3b, via a cable 6 so as to allow them to exchange power, image signals, control signals, and the like via the cable 6. The X-ray detection units 3a and 3b each function as a detector to detect X-rays transmitted through an object and obtain an X-ray image (radiographic image) based on the object. That is, the X-ray generating unit 4 and the X-ray detection units 3 cooperatively operate to implement X-ray imaging. Note that the X-ray detection units 3a and 3b are mounted on imaging tables 2, that is, 2a and 2b, in standing and lying positions.

An example of the arrangement of the X-ray imaging system has been described above. Note that the arrangement shown in FIG. 1 is merely an example, and can be changed as needed. For example, referring to FIG. 1, the respective apparatuses are connected to the X-ray imaging apparatus 10 via a communication unit 5. However, the X-ray imaging apparatus 10 need not always be connected to these apparatuses.

A procedure for processing to be performed when the X-ray imaging system shown in FIG. 1 captures an X-ray image along an examination procedure will be described below.

First of all, examination information is input to the X-ray imaging apparatus 10 in response to an examination request from the RIS 7. The examination information includes patient information including a patient name and patient ID and imaging information defining the contents of imaging to be executed for the patient.

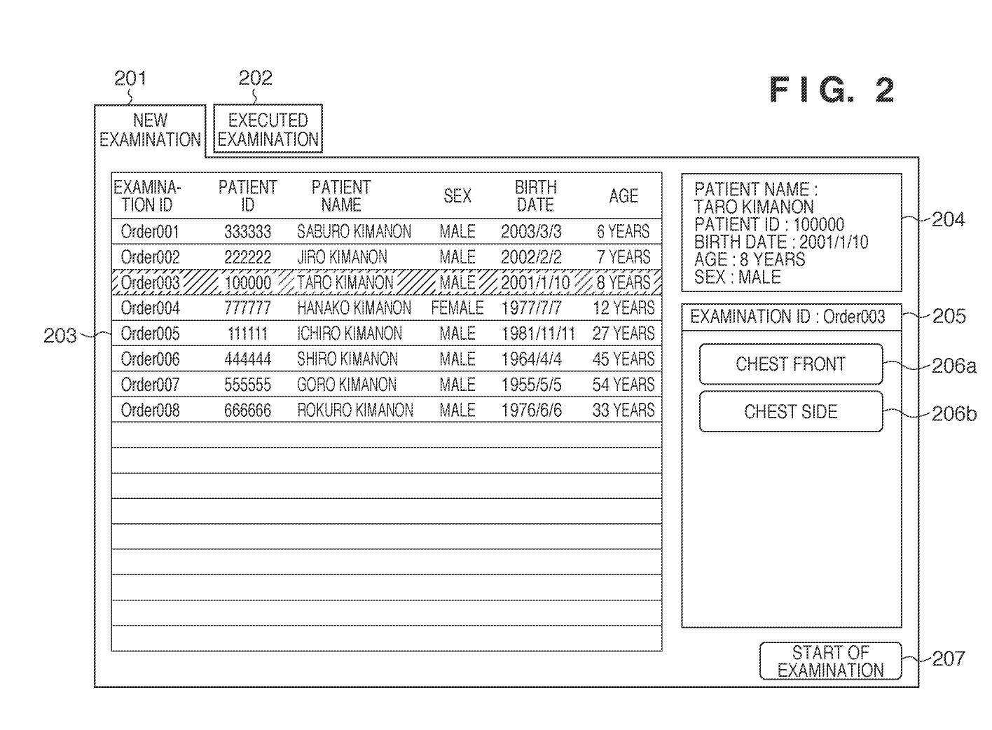

Upon receiving the examination information, the X-ray imaging apparatus 10 displays the new examination input screen shown in FIG. 2 on the display unit 11. As shown in FIG. 2, the new examination input screen includes a requested examination list 203, a patient information display area 204, an imaging information display area 205, and an examination start button 207.

In this case, the operator (imaging technician) selects any of the examinations in the requested examination list 203 by using the mouse or the like (operation unit 12). The display unit 11 then displays patient information (a patient ID, patient name, birth date, and the like) corresponding to the selected patient in the patient information display area 204. The display unit 11 also displays an examination ID in the imaging information display area 205, and imaging information corresponding to the examination ID in the area located immediately below the area 205. As described above, the X-ray imaging apparatus 10 receives imaging information from the RIS 7. The screen shown in FIG. 2 includes imaging method buttons 206, that is, a chest front button 206a and a chest side button 206b, corresponding to imaging information. The operator presses the examination start button 207 upon checking the patient information and the imaging information. This confirms the examination to be executed.

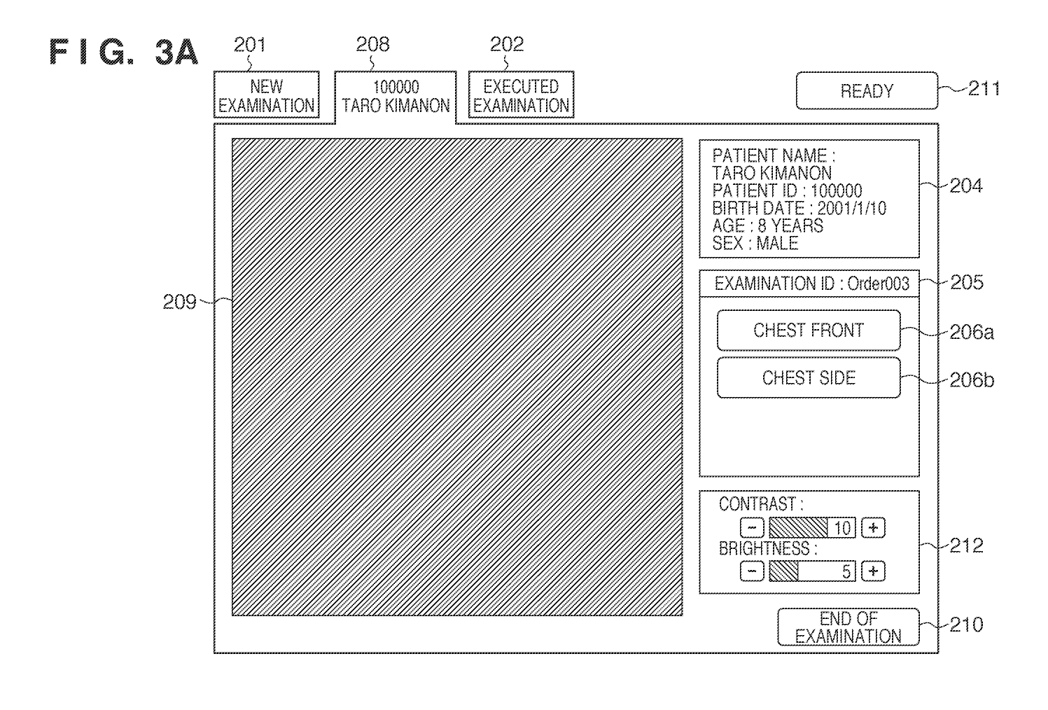

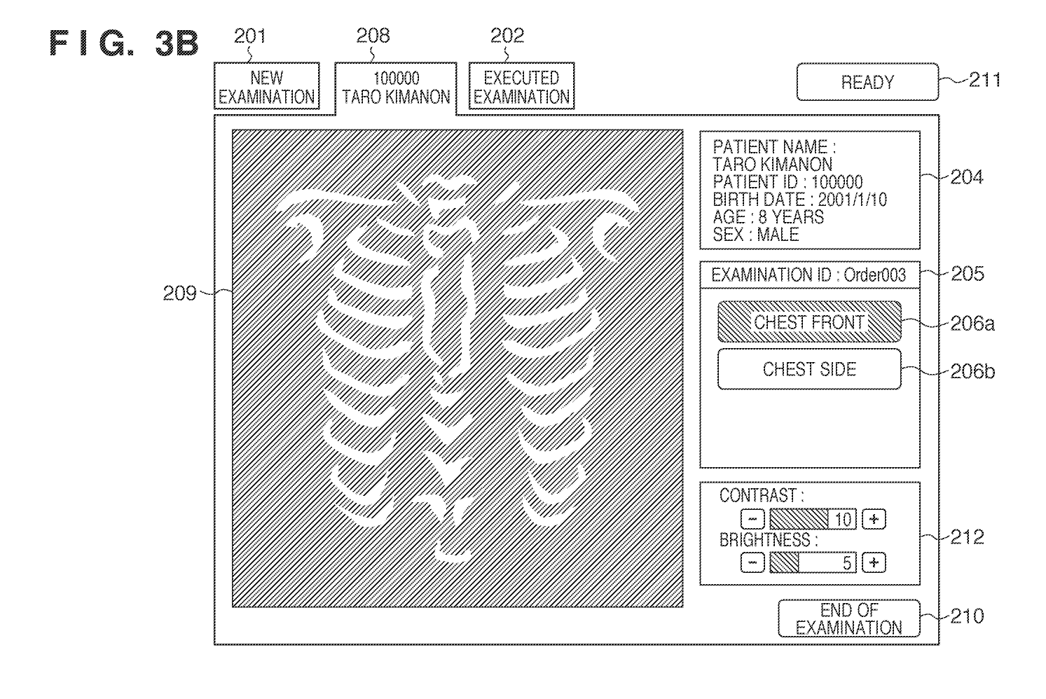

When the operator presses the examination start button 207, the X-ray imaging apparatus 10 displays the imaging screen shown in FIG. 3A on the display unit 11. The imaging screen is used at the time of imaging. If there is any examination that has already been executed, the X-ray imaging apparatus 10 closes the imaging screen corresponding to the examination under execution, and displays another imaging screen.

An imaging screen basically has the same display areas as those of the new examination input screen described with reference to FIG. 2. As shown in FIG. 3A, the display areas newly added to the imaging screen include an image display area 209, a patient information display area 204, an imaging information display area 205, a message area 211, and an image processing setting area 212.

When the imaging screen is displayed, the imaging method button 206a placed at the uppermost position in the imaging information display area 205 is set in a selected state by default. With this setting, the X-ray imaging apparatus 10 causes the control unit 13 to transmit imaging conditions (a tube voltage, tube current, irradiation period, and the like) set in accordance with the imaging method button (imaging method) to the X-ray generation control unit 14. The X-ray generation control unit 14 then controls the X-ray detection unit 3 in accordance with the imaging conditions to prepare for imaging.

Upon completing the preparation, the X-ray imaging apparatus 10 shifts to an imaging ready state. At this time, the X-ray imaging apparatus 10 displays a ready message indicating the imaging ready state in the message area 211.

Subsequently, the operator checks the imaging method and performs setting for imaging and positioning of the patient. Upon completing a series of imaging preparations, the operator refers to the message area 211 to determine that the apparatus is in the imaging ready state, and then presses an X-ray irradiation switch (not shown). The X-ray imaging apparatus 10 causes the X-ray generating unit 4 to irradiate the object (a specific region of the patient) with X-rays, and causes the X-ray detection unit 3 to detect the X-rays transmitted through the object. In this manner, the apparatus captures an X-ray image.

Upon completing the imaging process, the X-ray imaging apparatus 10 causes the control unit 13 to obtain a captured image from the X-ray detection units 3 and process the obtained captured image based on predetermined image processing conditions. The predetermined image processing conditions are defined in advance for the imaging method.

Upon completing the image processing, the X-ray imaging apparatus 10 displays the captured image having undergone the image processing in a captured image display area 209, as shown in FIG. 3B. When, for example, wanting to change the contrast or the like of the captured image, the operator operates buttons for contrast, brightness and the like which are provided in the image processing setting area 212. With this operation, the operator can execute additional image processing for the captured image displayed in the captured image display area 209. Note that when the operator presses the imaging method button 206 (206a, 206b) for regions which have not been imaged yet, the following imaging process starts.

The operator repeats the above procedure to execute imaging based on all the imaging methods in the imaging information display area 205. Upon completing all the imaging processes, the operator presses an examination end button 210. This terminates the series of examinations. The X-ray imaging apparatus 10 displays the new examination input screen again. At this time, the X-ray imaging apparatus 10 causes the control unit 13 to output captured images, examination information, and the like to, for example, the PACS 8, the printer (not shown), the RIS 7, the ROM in the apparatus itself, and the like. Note that the RIS 7, ROM, and the like store the captured images and the patient information in association with each other.

The following is a description of a procedure for operation to be performed when the operator checks an examination result in the X-ray imaging system shown in FIG. 1.

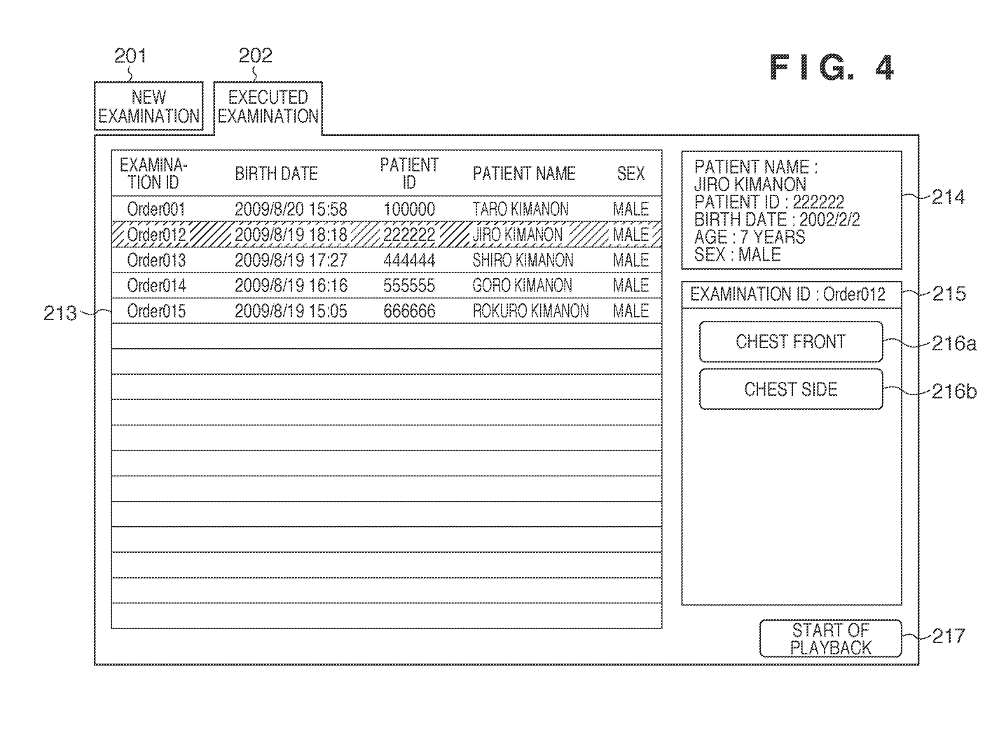

When the operator (doctor) performs a predetermined operation, the X-ray imaging apparatus 10 causes the display unit 11 to display the executed examination selection screen shown in FIG. 4. As shown in FIG. 4, the executed examination selection screen includes an executed examination list 213, a patient information display area 214, an imaging information display area 215, and a playback start button 217.

The display unit 11 displays a list of executed examinations in the executed examination list 213. The operator selects an examination as a playback target from the executed examination list 213 by the same operation as that on the new examination input screen described with reference to FIG. 2. The display unit 11 then displays patient information corresponding to the selected examination in the patient information display area 214. An imaging method button 216 corresponding to the imaging method at the corresponding examination time is placed in the imaging information display area 215. The operator presses the playback start button 217 after checking the patient information and the imaging information. This confirms the examination to be played back.

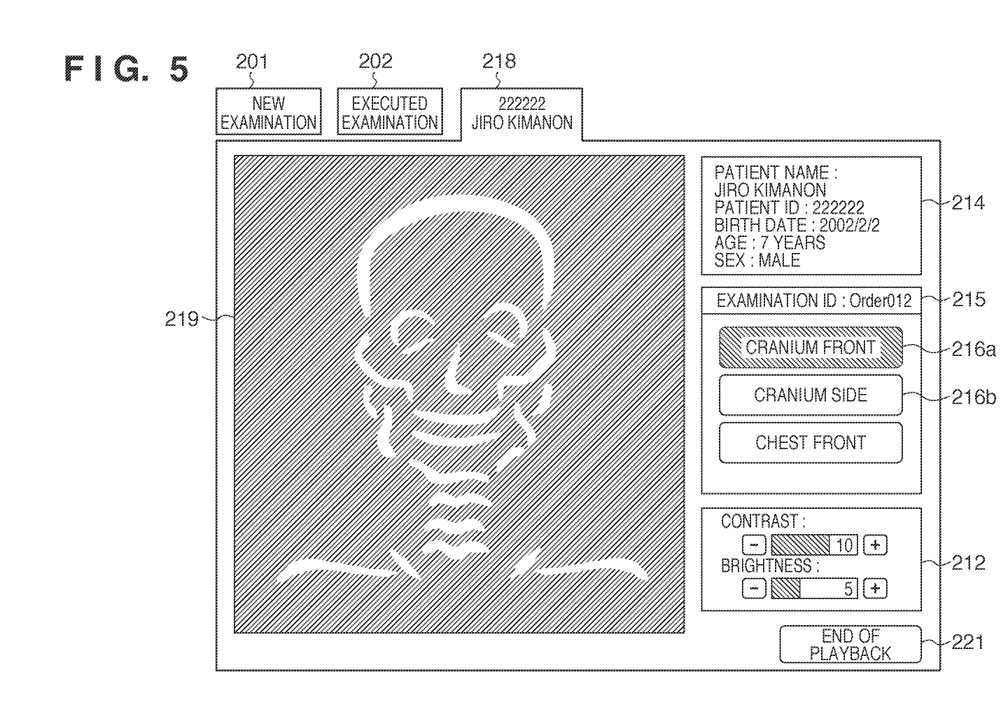

When the operator presses the playback start button 217, the X-ray imaging apparatus 10 causes the display unit 11 to display the playback screen shown in FIG. 5. The playback screen is used when an already captured radiographic image is to be played back. Note that if a playback screen is being displayed, the apparatus closes it and displays another playback screen.

A playback screen basically has the same display areas as those of the executed examination selection screen described with reference to FIG. 4. As shown in FIG. 5, the display areas newly added to the playback screen include an image display area 219 and an image processing setting area 212.

When the display unit 11 displays the imaging screen, an imaging method button 216a placed at the uppermost position in the imaging information display area 215 is set in a selected state by default. With this setting, the X-ray imaging apparatus 10 causes the control unit 13 to obtain the image captured in accordance with the selected imaging method button (imaging method) from a storage unit (for example, the ROM or PACS in the apparatus itself). With this operation, the display unit 11 displays the captured image in the image display area 219. When the operator presses a playback end button 221, the apparatus terminates the playback operation, and displays an executed examination selection screen.

All the screens described above, including the playback screen shown in FIG. 5, are provided with tab interfaces (to be simply referred to as tabs hereinafter). For example, the playback screen shown in FIG. 5 is provided with a new examination tab 201, an executed examination tab 202, and a playback examination tab 218. The operator can instantly switch and display the new examination input screen (see FIG. 2) and the executed examination selection screen (see FIG. 4) by operating the corresponding tabs using the mouse or the like (operation unit 12).

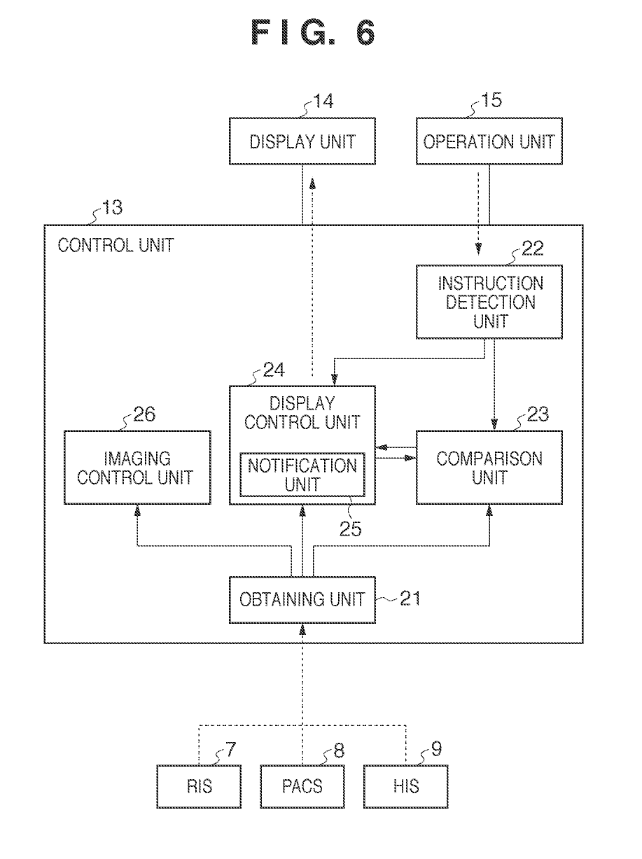

An example of the functional arrangement of the control unit 13 shown in FIG. 1 will be described next with reference to FIG. 6.

The control unit 13 includes an obtaining unit 21, an instruction detection unit 22, a comparison unit 23, a display control unit 24, and an imaging control unit 26 as components for controlling screen display.

The obtaining unit 21 obtains examination information and information necessary for screen display. For example, the obtaining unit 21 obtains captured images and the patient information associated with the images from the ROM, PACS 8, and the like in the apparatus itself. Note that captured images are stored in the ROM, the PACS 8, and the like in association with the patient information of the images. The obtaining unit 21 also obtains examination information based on an examination request from the RIS 7 from, for example, the ROM.

The imaging control unit 26 controls the X-ray imaging units (X-ray generating units 4a and 4b and the X-ray detection units 3a and 3b) in accordance with the examination information obtained by the obtaining unit 21. With this operation, the apparatus performs X-ray imaging based on the examination information.

The instruction detection unit 22 detects a display switching instruction for the currently displayed screen based on input operation by the operator. More specifically, when an imaging screen or playback screen is displayed and the operator operates a tab using the mouse or the like, the instruction detection unit 22 detects the operation and notifies the display control unit 24 and the comparison unit 23 of the issuance of a display switching instruction.

The comparison unit 23 compares information associated with the currently displayed screen with information associated with a switching destination screen in the execution of screen display switching (screen transition processing). Note that in this embodiment, information associated with a screen is patient information.

The display control unit 24 generates a display screen based on the examination information, captured image, and the like obtained by the obtaining unit 21. The display control unit 24 then displays the generated screen within one window using a tab on the display unit 11. When the instruction detection unit 22 detects a display switching instruction, the display control unit 24 switches the display of the screen on the display unit 11 in accordance with the instruction.

The display control unit 24 includes a notification unit 25 to perform notification (display) to call attention to the operator based on the comparison result obtained by the comparison unit 23. Assume that while a playback screen associated with an executed examination on patient A is displayed, the operator has input an instruction to switch to an imaging screen for the start of a new examination on patient B. That is, the pieces of patient information associated with the two screens differ from each other. In this case, the comparison unit 23 outputs a comparison result indicating that the pieces of patient information are different from each other, at the time of switching to the imaging screen. The display control unit 24 (notification unit 25) performs notification to call the attention of the operator based on the comparison result.

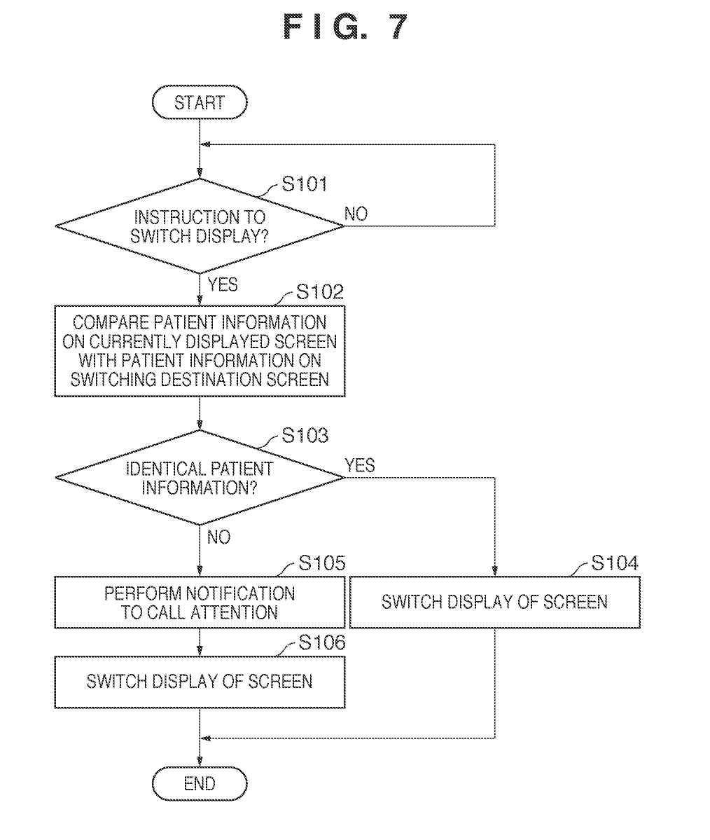

A procedure of processing at the time of notification to call the attention of the operator in the X-ray imaging system shown in FIG. 1 will be described with reference to FIG. 7. In this case, assume that after playing back an executed examination on a playback screen, the operator has issued an instruction to display a new examination input screen by using the new examination tab 201. The following is a description about processing after the operator has selected an examination as an execution target on the screen and the examination has started.

At the start of the examination, the X-ray imaging apparatus 10 causes the instruction detection unit 22 to detect a display switching instruction for the currently displayed screen (YES in step S101). The X-ray imaging apparatus 10 then causes the comparison unit 23 to compare the patient information associated with the examination played back on the playback screen with the patient information associated with the started examination (the imaging screen) (S102).

In this case, if the pieces of patient information (patient IDs, patient names, sexes, birth dates, and the like) coincide with each other (YES in step S103), the X-ray imaging apparatus 10 causes the display control unit 24 to directly switch the display to the imaging screen (S104).

If the pieces of patient information differ from each other (NO in step S103), the X-ray imaging apparatus 10 causes the notification unit 25 to notify the user that the apparatus has started an examination on the patient different from the one corresponding to the currently played back examination (S105). The display control unit 24 switches the display to an imaging screen (S106).

A concrete example of the notification method used in step S105 in FIG. 7 will be described below.

In some general examinations and the like, while an executed examination result on a given patient is played back, a new examination on another patient is started. Such cases include, for example, a case in which the apparatus refers to images of identical regions of different patients with similar physiques to use image processing parameters for one patient as those for the other patient. For this reason, in some cases, even when the operator starts an examination on a patient different from the patient currently played back on a playback screen, there is no need to perform notification to call attention to the operator.

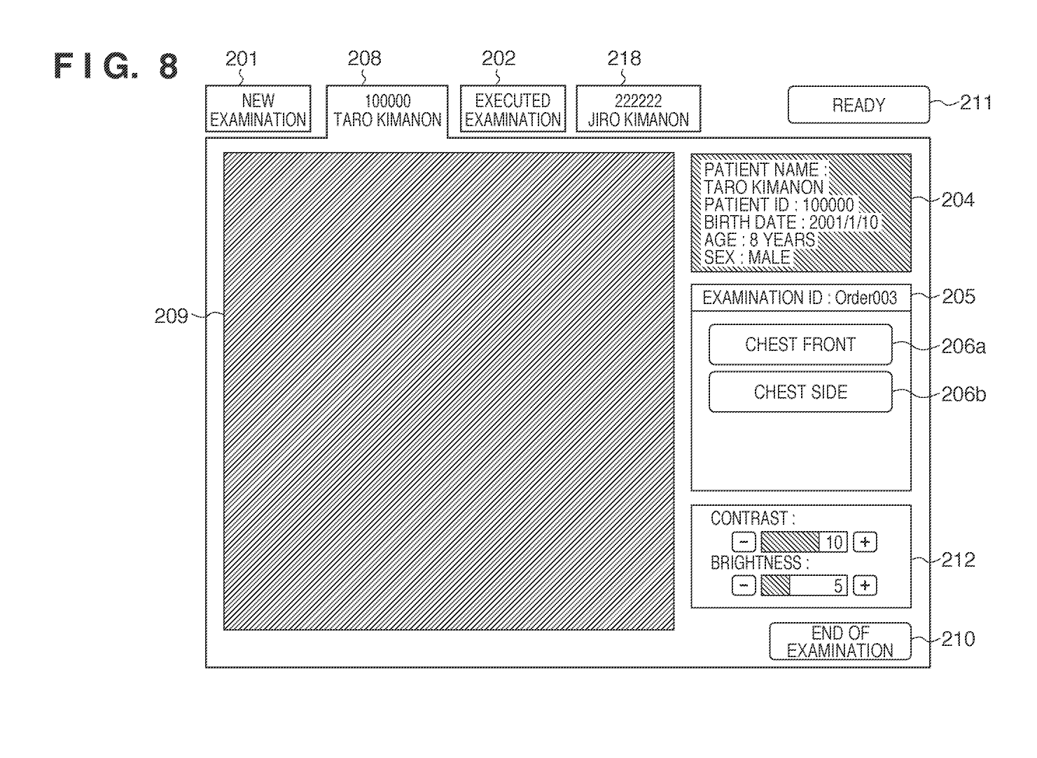

For example, as shown in FIG. 8, the first embodiment therefore performs notification by changing the background color of the patient information display area 204 for a predetermined period of time instead of using a notification scheme of requesting the operator to perform operation to cancel a notified state (using, for example, a modal dialogue). That is, the embodiment performs notification by changing a display form.

Although not described here, it is possible to perform notification similar to that described above also in a case in which while an examination is executed, an executed examination result on another patient is played back. That is, it is possible to compare pieces of patient information at the time of transition to a playback screen and perform notification if the patients differ from each other.

As described above, the first embodiment can effectively prompt the operator to check patients at the time of display switching. If, for example, the operator erroneously operates to start an examination on a patient different from a currently played back patient, this apparatus performs notification to call the attention of the operator for a predetermined period of time at the time of display switching. This can reduce the risk of mixing up of the patients by the operator.

### Second Embodiment

The second embodiment will be described next. The first embodiment has exemplified the case in which when the operator presses the examination start button 207 on the new examination input screen shown in FIG. 2, if an examination has already been executed, the imaging screen corresponding to the examination under execution is closed. In contrast to this, the second embodiment will exemplify a case in which when the operator presses an examination start button 207, if an examination has already been executed, a new imaging screen is generated and displayed without any suspension of the examination. Note that a playback screen in the second embodiment is the same as that in the first embodiment.

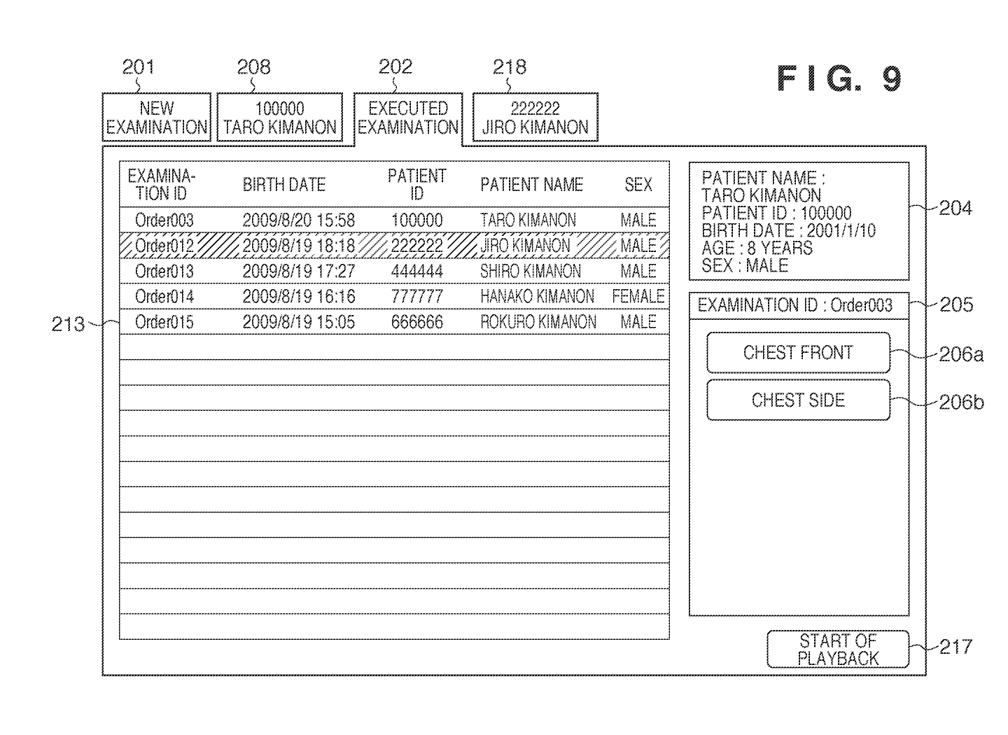

FIG. 9 is a view showing an example of an executed examination selection screen according to the second embodiment. The following is a description about a case in which an imaging screen and a playback screen are associated with tabs, and the operator has issued an instruction to display new imaging screen using new tabs.

Assume that the operator has selected an examination on the same patient as the one on the currently displayed playback screen (the playback screen which has already been associated with a tab) from an executed examination list 213, and has pressed a playback start button 217. An X-ray imaging apparatus 10 then causes a display control unit 24 to generate a new playback screen and display the playback screen while associating it with a new tab. At this time, the X-ray imaging apparatus 10 causes a comparison unit 23 to compare the patient information associated with all examinations that are being executed and played back with the patient information on the playback screen to be newly played back.

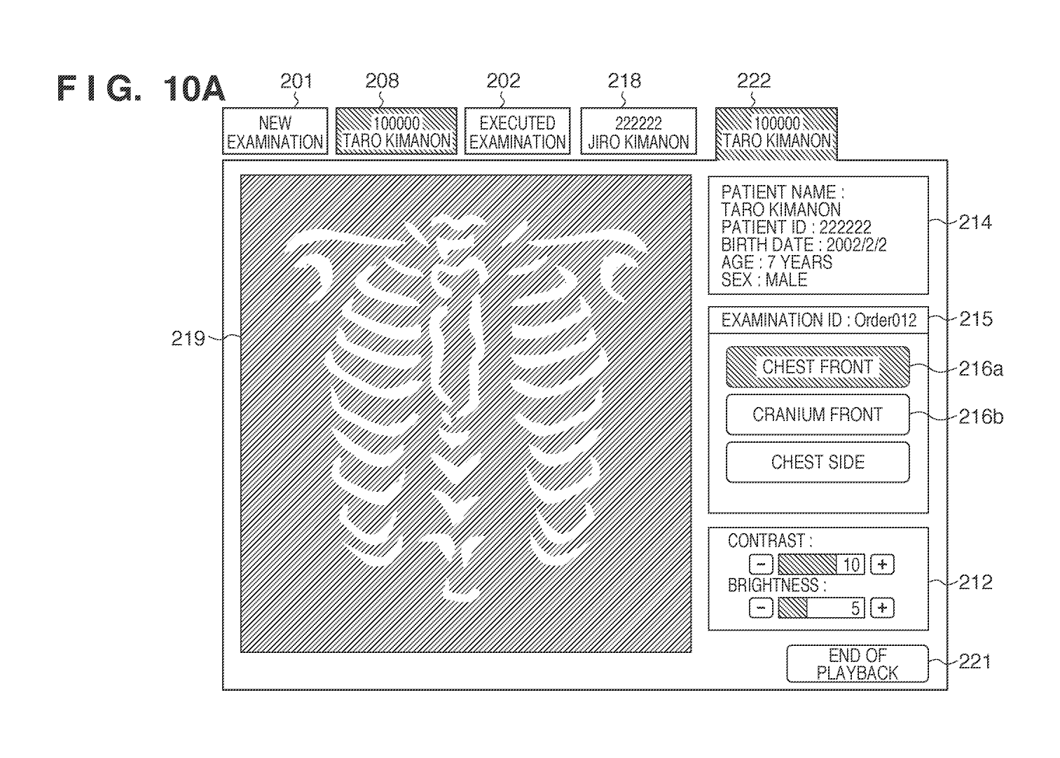

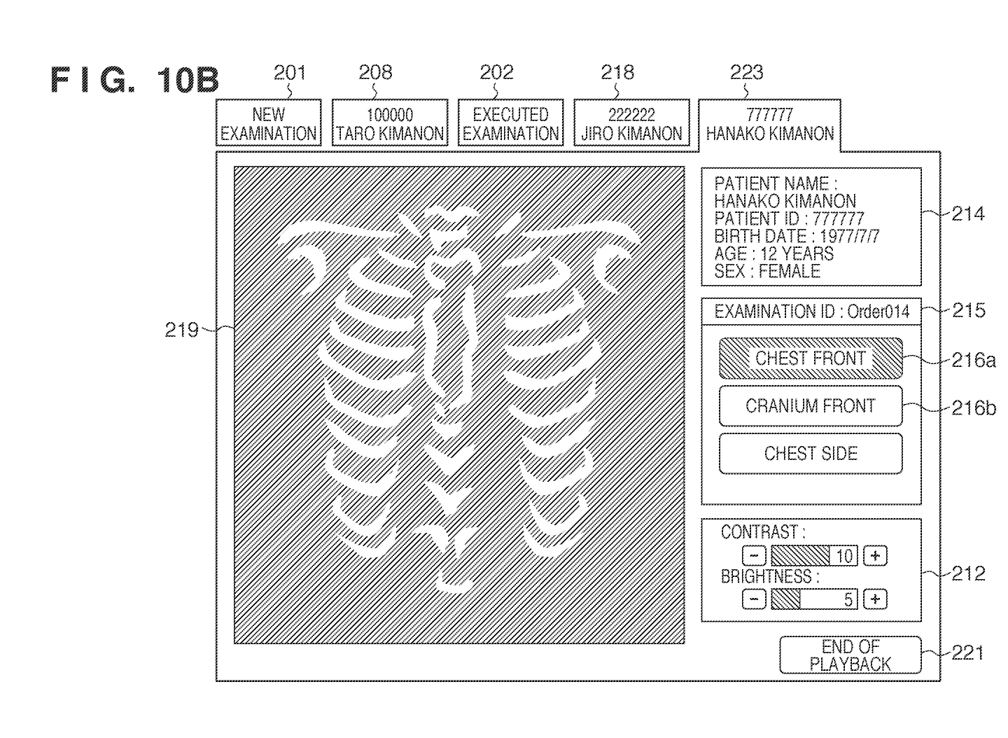

If the comparison result indicates any screen handling the same patient, this apparatus changes the display form of tabs associated with the corresponding screen (in this case, tabs 208 and 222), as shown in FIG. 10A, to notify the operator of the presence of an examination executed on the same patient. In contrast, if there is no screen handling the same patient, the apparatus generates a new tab and directly displays a playback screen on the tab without any notification, as shown in FIG. 10B.

The second embodiment has exemplified the case in which when an imaging screen and a playback screen are displayed in associated tabs, another imaging screen is newly displayed. Obviously, however, even when another playback screen is to be displayed, processing similar to that described above can be performed.

In addition, when a plurality of tabs with which an imaging screen or a playback screen is associated is displayed, and the operator has designated any of the tabs, it is possible to perform processing similar to that described above. When, for example, an imaging screen is associated with one tab while a playback screen is associated with two tabs, and the operator has designated any of the tabs, it is possible to compare patient information on the screen associated with the designated tab with patient information on the remaining screen and notify the result. Furthermore, referring to FIGS. 10A and 10B, tabs are provided on an examination basis. However, tabs may be provided on a patient basis.

As described above, the second embodiment can obtain the same effects as those of the first embodiment and concurrently execute a plurality of examinations.

The typical embodiments of the present invention have been described above. However, the present invention is not limited to the embodiments described above and shown in the accompanying drawings, and can be modified and executed as needed within the spirit and scope of the invention.

For example, the first and second embodiments have exemplified the case in which notification is performed by changing the display form of a screen. However, the present invention is not limited to this. For example, it is possible to call the attention of the operator by sounding an alarm or the like.

In addition, the present invention can take embodiments as a system, apparatus, method, program, storage medium, and the like. More specifically, the present invention can be applied to a system including a plurality of devices, or to an apparatus including a single device.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2010-036666 filed on Feb. 22, 2010, which is hereby incorporated by reference herein in its entirety.

What is claimed is:

1. A radiation imaging system which captures a radiographic image, the system comprising:
an obtaining unit configured to obtain examination information including patient information of a patient as a target for the imaging and imaging information defining a content of imaging executed for the patient;
an imaging control unit configured to control imaging of a patient using radiation in accordance with examination information obtained by said obtaining unit;
a storage unit configured to store the captured radiographic image in association with the patient information of the image;
a display unit configured to switch and display, within one window, an imaging screen used for imaging based on the examination information and a playback screen used for playback of a radiographic image stored in said storage unit;
an instruction detection unit configured to detect a switching instruction for display of a screen displayed on said display unit, based on input operation by an operator;
a comparison unit configured to compare patient information corresponding to the imaging screen with patient information corresponding to the playback screen when display switching is performed based on the switching instruction; and
a notification unit configured to notify the operator of a comparison result obtained by said comparison unit.

2. The system according to claim 1, wherein if the comparison result obtained by said comparison unit indicates that the pieces of patient information differ from each other, said notification unit notifies of corresponding information.

3. The system according to claim 1, wherein said display unit displays tabs associated with the imaging screen and the playback screen in the window,
said instruction detection unit detects, upon issuance of an instruction to generate a new tab and a new imaging screen or a new playback screen which is associated with the tab, the instruction as the switching instruction,
said comparison unit compares patient information corresponding to the new screen with patient information corresponding to all screens associated with the tab at the time of switching of display to the new screen, and
if a comparison result obtained by said comparison unit indicates that there is a screen having patient information identical to patient information corresponding to the new screen, said notification unit notifies of corresponding information.

4. The system according to claim 1, wherein said display unit displays tabs associated with the imaging screen and the playback screen in the window,
said comparison unit compares, at the time of switching of display to a screen associated with a tab designated by input operation by the operator, patient information corresponding to the designated screen with patient information corresponding to other screens, and
if a comparison result obtained by said comparison unit indicates that there is a screen having patient information identical to patient information corresponding to the designated screen, said notification unit notifies of corresponding information.

5. The system according to claim 3, wherein said notification unit performs the notification by changing a display form of a tab associated with the screen having the identical patient information.

6. The system according to claim 1, wherein said notification unit performs the notification by changing a display form of the patient information.

7. The system according to claim 1, wherein said notification unit performs the notification by sounding an alarm.

8. The system according to claim 1, wherein said notification unit performs the notification for a predetermined period of time.

9. A processing method in a radiation imaging system which captures a radiographic image, the method comprising:
obtaining examination information including patient information of a patient as a target for the imaging and imaging information defining a content of imaging executed for the patient;
controlling imaging of a patient using radiation in accordance with the obtained examination information;
storing the captured radiographic image in association with the patient information of the image;
switching and displaying, within one window, an imaging screen used for imaging based on the examination information and a playback screen used for playback of the stored radiographic image;
detecting a switching instruction for display of the displayed screen based on input operation by an operator;
comparing patient information corresponding to the imaging screen with patient information corresponding to the playback screen when display switching is performed based on the switching instruction; and
notifying the operator of the comparison result.
